(19) United States
(12) Patent Application Publication
(10) Pub. No.: US 2024/0358590 A1
(43) Pub. Date: Oct. 31, 2024

(54) ZERO MAGNETIC FIELD COIL FOR ACUPUNCTURE POINTS

(71) Applicant: MS CORPORATION CO., LTD., Fukuoka-shi, Fukuoka (JP)

(72) Inventors: Keiichi AKAZAKI, Fukuoka-shi, Fukuoka (JP); Nobuyuki KOBAYASHI, Komoro-shi, Nagano (JP); Nobumori KOBAYASI, Ueda-shi, Nagano (JP)

(21) Appl. No.: 18/561,092

(22) PCT Filed: Mar. 30, 2022
(86) PCT No.: PCT/JP2022/015990

§ 371 Date: Nov. 15, 2023

(30) Foreign Application Priority Data
May 18, 2021 (JP) 2021-083531

(51) Int. Cl. A61H 39/04; A61H 39/00; A61N 2/02

(52) U.S. Cl. CPC A61H 39/04; A61H 39/002; A61N 2/02

(57) ABSTRACT

An acupuncture point stimulation coil to constitute a zero magnetic field includes individual members, each having a spiral portion wound in a left-winding direction and another spiral portion wound in a right-winding direction, the spiral portion and the another spiral portion being vertically combined to be one unit, or each having at least two spiral portions wound in the left-winding direction and at least two another spiral portions wound in the right-winding direction, the at least two spiral portions and the at least two another spiral portions being vertically combined, respectively, to be at least two units, and at least one conductive wire continuously extending, each connecting two of the at least two units.

### FIELD OF THE INVENTION

The present disclosure relates to a novel coil that is an electroconductive acupuncture point stimulation coil to constitute a zero magnetic field and that contributes to maintenance of homeostasis on a basis of theory particularly discovered from standpoints of beauty and healthcare. For further details, the present disclosure relates to the coil and a method for using the same wherein very weak current of pulse wave generated by a peculiar coil structure moderately stimulates capillaries in a human body to normalize blood flow to thereby elicit a natural resilience inherently possessed in humans.

### BACKGROUND OF THE INVENTION

We daily act such as opening and closing our eyes and noses, and drinking, eating, blowing, and talking with our mouths, etc., and then muscular movement of a head or a face and maintenance of normalized blood flow cause firmness on a face skin, where aging is noticeable, and a variety of facial expressions.

Here, muscle of the head is classified into mastication muscle to move a lower jaw and mimetic muscle to create facial expressions, and the mimetic muscle is a muscle, as its name, that creates the variety of facial expressions such as delight, anger, sorrow and pleasure.

Further, the mimetic muscle is controlled by facial nerves; however, the mimetic muscle has a function to sense one's own “emotion” and how the “emotion” is related to next act to others, and it is one of outstanding humans' abilities, as compared with animal species.

On the other hand, orbicularis oris muscle is a muscle that surrounds a mouth, and it is mainly involved in movement of a lip and affects movement of closing and pursing the mouth. Various mimetic muscles are attached to the orbicularis oris muscle, and the orbicularis oris muscle is the central muscle of the face such that 70 percent of the mimetic muscle is around the mouth.

Furthermore, because we consciously move the orbicularis oris muscle, corners of the mouth droop and wrinkles increase due to aging; as a result, a face line collapses and impression from the face is significantly changed.

The orbicularis oris muscle and the mimetic muscle tend to decline with age; however, there is individual differences, and a primary factor is to normalize blood flow, as mentioned at the beginning.

The blood flow is particularly important from a medical view, and from Oriental medicine view, for example, in traditional Chinese medicine, a status of human health, i.e. “Proof”, is measured by considering balance among “Energy”, “Blood” and “Water”, and a state of the “Blood” in a human's body is considered as an extremely important factor because therapeutic agent is prescribed according to the status.

As for this point, if it is broadly considered as the balance of the blood flow in an entire human body (capillaries and organization role), the “Blood” is an extremely important factor that is unable to be overlooked from an esthetics view besides the medical view.

Especially, in the recent coronavirus pandemic, in living environment wherein we have a habit to wear masks in our lifestyle, it is concerned that “old-looking face” proceeds unconsciously. For example, as a mask is worn for longer time, in covered and contacted areas, skin troubles such as itching, rashes, pigmentation, and acne are occurred, and even such change of the lifestyle is in an unprecedented state.

Here, for considering wearing a mask once again, it is a major issue on beauty that due to wearing the mask everyday, we lost opportunity to move the “mimetic muscle” and the muscle and its movement function are weakened, thereby the blood flow of the face is worse.

A secret key of so-called “beautiful face” is to improve the blood flow in the capillaries presented in the entire face. If skin cells are activated and metabolism of skin surface is improved, old keratin is smoothly removed. Simultaneously, the movement function such as the mimetic muscle is normalized. In this way, if cell regeneration cycle and functions of muscular tissues are normalized, skin rejuvenation and aging prevention naturally occur. Furthermore, the “Water” is closely related to moisturizing condition of skin and is essential to normalize the blood flow; however, water is required to supply into a body if the body lost the water. It is also important for skin hydration to maintain the balance by applying an external preparation besides supplying the water into the body.

As stated above, a problem of the blood flow needs to be broadly considered from a view of homeostasis that humans inherently have, and because it is a very important element in medical and cosmetic views, attempts to improve the blood flow have been made for a long time. For example, there are Patent Documents 1 to 4.

In Patent Document 1, an eye mask equipped with magnets is disclosed. The eye mask is made of cloth, paper, and chemical fiber with the magnets, and it is a health product designed to remove fatigue without medicine by relieving stiffness and improving blood flow.

In Patent Document 2, a technique related to an instrument for beauty treatment with utilizing magnets is disclosed. The instrument is designed to include several permanent magnet pieces adjustably attached on an inside surface of an instrument body having a shape such as a frame of eyeglasses, and blood flow of skin tissue in a face is improved and beauty treatment is brought on the face.

In Patent Document 3, a skin care sheet for caring skin, placed on a face, neck, etc., is disclosed. The skin care sheet includes a conventional sheet body impregnated with a beauty essence and magnetism to improve its effects. Accordingly, the skin care sheet is provided to activate blood flow in capillaries and to improve metabolism by giving appropriate stimulation to skin, muscles, and nervous system through the magnetism in the skin sheet, and to effectively infiltrate the beauty essence into the skin through skin pores by burning fat and floating deteriorated substances on the skin.

In this way, in all of those Patent Documents 1 to 3, the magnetism is applied as the technique for improving the blood flow, and each technique has its own feature; however, it has only partial effect and sustainability is insufficient.

For considering the sustainability, there is a limit to utilizing magnets externally added, and Applicant, rather, attempts to consider utilization of energy inherently existing in a human in relation to blood flow by reversal thinking of the utilization of energy externally added. Accordingly, as disclosed in Patent Document 4 in which Applicant's invention is described, even though it is an application of generating weak current (electromotive force) in a living body, by conducting research based on assumption that abnormal blood flow is caused by retention of the current in the living body, it is achieved to improve the blood flow with a specific coil and to provide a coil with a special structure to be useful in a medical field.

The particular coil has a structure including two coils wherein one of the two coils winded in a reversed direction, and magnetic forces generated by the weak current in the living body are canceled each other. Then, because no external current is applied, magnetic fields based on Fleming's left-hand rule are canceled each other, and a zero magnetic field is formed. In a single coil, because a right-winding coil and a left-winding coil are combined, the current flowing through each coil is canceled to be equilibrium; however, because internal energy is latently contained, the blood flow is improved.

As for this point, as shown in inference, various medical effects are recognized due to improvement of the blood flow; however, effects against chronic symptoms are not sufficiently recognized. This result shows that it sufficiently affects against partial blood retention. On the other hand, it does not affect against sustainability, and it is demanded to provide further improvements and a method for using the existing coil.

### PATENT DOCUMENTS

### Patent Document 1:

Japanese Utility Model Application Publication No. Showa 57-70334
Patent Document 2:
Japanese Utility Model Application Publication No. Showa 58-92953
Patent Document 3:
Japanese Utility Model Registration No. 3101446
Patent Document 4:
Japanese Patent Application Publication No. 2010-29518

### Problems to be Solved by the Invention

The present disclosure is invented in view of the above unsolved problem. The present disclosure relates to a novel coil that is an electroconductive acupuncture point stimulation coil to constitute a zero magnetic field and that contributes to maintenance of homeostasis on a basis of theory particularly discovered from standpoints of beauty and healthcare; and provides a coil and a method for using the same wherein very weak current of pulse wave generated by a peculiar coil structure moderately stimulates capillaries in a human body to normalize blood flow to thereby elicit a natural resilience inherently possessed in humans.

### Means for Solving the Problems

The inventors aim for developing a coil that further contributes to the sustainability while taking advantage of the features disclosed in Patent Document 4. It is a remarkable that the zero magnetic field coil, disclosed in Patent Document 4, is integrally formed with a left-winding coil and a right-winding coil, and improves the blood flow and relieves pain in affected areas even though the weak currents generated by the left-winding coil and right-winding coil are canceled each other.

Thus, after carefully considering mechanism of the weak current mediation in the clinical result, it is recognized that the weak currents are canceled each other; however, it does not mean that any current is not flowing in chronological order, and the currents flow through the left-winding coil and the right-winding coil, as pulse waves, and are constantly canceled each other on the skin.

Due to this, it is assumed that source of the blood flow promotion is energy generated by the pulse waves. As shown in FIG. 3, the generation and cancellation of the pulse wave currents are repeated on micro scale. On the other hand, density of magnetic flux based on the Fleming's left-hand rule also repeats the generation and cancellation. It is assumed that stimulation to skin surface is caused by interaction between the pulse wave currents and the energy generated by the magnetic flux; however, conditions for improving the sustainability are not found.

As for this point, even though the coils disclosed in Patent Document 4 are randomly (arbitrarily) arranged regardless of acupuncture points, in the method disclosed in Patent Document 4, ununiformed blood flow are caused and heat is sensed, and further the affected areas are reddish because the energy is partially concentrated and the heat is supplied all at once. Accordingly, it seems that there is a lack of overall sustainability because efficiency of the heat energy is not sufficient and the heat energy is wasted. Therefore, the inventors conclude that countermeasure against this is a key to improve the sustainability, and by improving coil arrangement (stimulation at acupuncture points) and structure, the inventors continuously attempt optimizing the overall energy and eventually invent the novel coil.

Here, in the present disclosure, as an embodiment of a single coil disclosed in Patent Document 4, each coil is arranged at one acupuncture point or more than two coils are arranged at the acupuncture point depending on an usage area. Accordingly, the coils may be arranged at optimum positions to spread the blood flow entirely. As a particular matter, in addition that the acupuncture points are different from each other depending on the usage areas, the optimum positions are moved by individual differences; therefore, it is preferable to select and process the number of coils and optimum materials to be applied to each individual.

As improvement of the coil itself, preferably, more than two of a single conductive coil to constitute the zero magnetic field are consecutively arranged on a single conductive wire so that the energy generated by the weak currents in the human body and the energy generated by the magnetic flux are distributed without any waste.

In this way, the zero magnetic field coil related to the present disclosure is invented to match a novel arrangement of the existing coils (independent configuration type) or a novel structure of more than two of the single coil with a theory of the acupuncture points, and improvement of the blood flow with desired sustainability is achieved by applying the coil having the novel structure. As a method for using the single existing coil, the single coil may be arranged at one acupuncture point or more than two of the single coil may be arranged in order to partially stimulate the acupuncture point. Further, in a case that more than two of the single coil are arranged, if the coils are connected through a single conductive wire, it is convenient for an entire application; then, it may be appropriately selected depending on the usage areas. As one formation example of two of the single coil is shown in FIG. 4. Two coil type in the present disclosure is the one in which each single coil shown in FIG. 4 is connected through the single conductive wire, and the number of coils may be freely selected depending on a place of the acupuncture point, application location, and intended use because more than n (n is an integer of 2 or more) coils may be arbitrarily included.

In the present disclosure, the coil to constitute the specific zero magnetic field is provided wherein the weak currents in the human body are utilized as an energy source, and the bioenergy is circulated in an entire body to maintain the blood flow without any waste in order to normalize homeostatic function of the human body; and the present disclosure broadly includes other embodiments that change formation in easy to use as explained later.

Furthermore, the conductive wire in the present disclosure is preferably made of copper having excellent thermal conductivity and processability with antibacterial properties; however, a heat amount may be excessive depending on the usage areas, and in that case, for example, aluminum may be selected. In order to prevent excessive energy by directly contacting the affected areas, coating with a support body made of urethane or silicone material may also be appropriately selected. As a material for adjusting magnetic field energy of the zero magnetic field coil, platinum silicone, carbon or tourmaline may be appropriately selected. Carbon and tourmaline are useful for adjusting the magnetic field, particularly enhancing the magnetic field environment, while increasing generation of the pulse wave currents, because carbon and tourmaline are conductive materials; however, in any case, those contributions are in the equilibrium constituting the zero magnetic field that is the essence of the present disclosure. Moreover, it may be difficult to be processed into application products depending on the usage. For example, it is difficult to be processed into textile products because mass production is had to be achieved due to high labor and cost for embedding coils, and there is a weight reduction problem even in finished products. As for this point, particularly, if field athletes wear the products, it goes against the needs to reduce weight by even 1 g. In that case, as one of methods for applying to wide range of the usage and for easily providing products without losing any effects of the zero magnetic field coil, conductive ink is utilized. More specifically, as one example, a shape to have effects same as the zero magnetic field coil is drawn in the specific support body with the conductive ink.

Accordingly, the present disclosure provides the electroconductive acupuncture point stimulation coil to constitute the zero magnetic field and the method for using the same, as described below.
(1) the acupuncture point stimulation coil to constitute the zero magnetic field, including a single conductive wire continuously extending, and individual members, each having a spiral portion wound in a left-winding direction and another spiral portion wound in a right-winding direction, the spiral portion and the another spiral portion being vertically combined to be one unit, wherein two of the individual members are arranged or two or more of the individual members are continuously arranged. (2) the acupuncture point stimulation coil to constitute the zero magnetic field according to (1), wherein the conductive wire is made of copper or aluminum.
(3) the acupuncture point stimulation coil to constitute the zero magnetic field according to (1) or (2), wherein the acupuncture point stimulation coil is covered with a silicone cover. (4) the acupuncture point stimulation coil to constitute the zero magnetic field according to any one of (1) to (3), wherein the silicon cover is kneaded or coated with platinum silicone, carbon, or tourmaline.
(5) the acupuncture point stimulation coil to constitute the zero magnetic field according to any one of (1) to (4), wherein if the coil includes one individual member, arbitrary number of the coils are discontinuously arranged on the support body arbitrarily formed along acupuncture points, and if the coil includes two or more individual members, the coils are arranged depending on a shape of the support body arbitrarily formed along the acupuncture points.
(6) the acupuncture point stimulation coil to constitute the zero magnetic field according to (5), wherein the support body is made of urethane or silicone.
(7) the acupuncture point stimulation coil to constitute the zero magnetic field according to any one of (5) to (6), wherein the support body is kneaded or coated with platinum silicone, carbon, or tourmaline.
(8) a method for using the acupuncture point stimulation coil to constitute the zero magnetic field, including a step of appropriately arranging at least one individual member to improve blood flow wherein the acupuncture point simulation coil includes a single conductive wire continuously extending, and the at least one individual member, each having a spiral portion wound in a left-winding direction and another spiral portion wound in a right-winding direction as one basic unit, the spiral portion and the another spiral portion being vertically combined.

### Effects of the Invention

The acupuncture point simulation coil, in the present disclosure, includes a single conductive wire continuously extending, and individual members, each having a spiral portion wound in a left-winding direction and another spiral portion wound in a right-winding direction as one basic unit, the spiral portion and the another spiral portion being vertically combined; and at least one individual member is independently formed or arranged, or two or more of the individual members are continuously included to be n (n is an integer of 2 or more) individual members. In the usage state of the coils, the coils are arranged at portions corresponding to the acupuncture points to cause appropriate blood flow due to energy generated by pulse wave currents and electromagnetic fields and to normalize circulation of stagnant blood. Therefore, various esthetics and medial troubles caused by blood retention in body parts having many capillaries and by disrupting biological balance due to abnormal blood flow are solved.

Here, the zero magnetic field coil in present disclosure has other interesting properties. Accordingly, it has effects of suppressing and eliminating static electricity. Generation of the static electricity is known as a factor causing blood flow disorders; however, elimination of the static electricity is considered as another factor that the coil contributes to improvement and normalization of the blood flow.

Further, there are ripple effects by the usage of the coil. The zero magnetic field coil has effects of suppressing and eliminating static electricity, as mentioned above; therefore, for example, in daily life, the coil may be equipped in a comb as one of daily supplies to remove tangled hair.

### BRIEF DESCRIPTION OF THE DRAWINGS

FIG. 1 is a basic structure of the zero magnetic field coil.

FIG. 2 is a combination in a basic unit of the coil.

FIG. 3 is a model of generation of the zero magnetic fields and the pulse waves in the coil.

FIG. 4 is the zero magnetic field coil wherein two basic units are arranged.

FIG. 5 is a prototype of an eye mask.

FIG. 6 is a prototype of a face mask.

FIG. 7 is an example of fitting the eye mask on eyes.

FIG. 8 is an example of mounting the face mask on a face.

FIG. 9 is a front view of a product design of the face mask.

FIG. 10 is a rear view of the product design of the face mask.

FIG. 11 is a side view of the product design of the face mask, a right side view and a left side view being symmetric to each other.

FIG. 12 is a top plan view of the product design of the face mask.

FIG. 13 is a bottom plan view of the product design of the face mask.

FIG. 14 is an example of mounting the face mask (band fitting type) on a face (a front view).

FIG. 15 is an example of mounting the face mask (band fitting type) on a face (a side view).

FIG. 16 is a basic unit of the coil.

FIG. 17 is the coil embedded in a support body.

FIG. 18 is an action for increasing an amount of blood flow.

FIG. 19 is a measuring method of an action for suppressing positive ion.

FIG. 20 is a measurement result of an action for suppressing positive ion.

Hereafter, embodiments of the acupuncture point stimulation coil to constitute the zero magnetic field will be described based on the drawings; however, the scope of the present disclosure is not limited to the embodiments.

As mentioned above, the present disclosure relates to a novel coil that is an electroconductive acupuncture point stimulation coil to constitute a zero magnetic field and a method for using the same that contributes to maintenance of homeostasis on a basis of theory particularly discovered from standpoints of beauty and healthcare. Weak current of pulse wave generated by a peculiar coil structure moderately stimulates capillaries at the acupuncture points (361 holes in oriental medicine) in a human body to normalize blood flow to thereby elicit a natural resilience inherently possessed in humans. In the oriental medicine, it is one of particularly important medical treatments to selectively stimulate 361 holes existed in a human body, and the treatment system is based on “mind and body as one.” Here, the “mind and body as one” is a treatment system based on a though in which mind and body are strongly interacted to each other, and for example, it is pathologically proved that stimulation to the acupuncture points is affected in a brain and a frontal lobe is stimulated; thereby it can be a countermeasure against depression. In order to maintain the balance, orexin produced in the hypothalamus is involved. Orexin is a neuropeptide that plays an important role in the functional balance between sleep and wakefulness, and is an acidic substance in a human body for maintenance of homeostasis. Therefore, improvement of the blood flow due to the coil is expected to cause ripple effects to normalize mentality. Accordingly, the present disclosure provides:
(1) the acupuncture point stimulation coil to constitute the zero magnetic field, including a single conductive wire continuously extending, and individual members, each having a spiral portion wound in a left-winding direction and another spiral portion wound in a right-winding direction, the spiral portion and the another spiral portion being vertically combined to be one unit, wherein two of the individual members are arranged or two or more of the individual members are continuously arranged.
(2) the acupuncture point stimulation coil to constitute the zero magnetic field according to (1), wherein the conductive wire is made of copper or aluminum.
(3) the acupuncture point stimulation coil to constitute the zero magnetic field according to (1) or (2), wherein the acupuncture point stimulation coil is covered with a silicone cover. (4) the acupuncture point stimulation coil to constitute the zero magnetic field according to any one of (1) to (3), wherein the silicon cover is kneaded or coated with platinum silicone, carbon, or tourmaline.
(5) the acupuncture point stimulation coil to constitute the zero magnetic field according to any one of (1) to (4), wherein if the coil includes one individual member, arbitrary number of the coils are discontinuously arranged on the support body arbitrarily formed along acupuncture points, and if the coil includes two or more individual members, the coils are arranged depending on a shape of the support body arbitrarily formed along the acupuncture points.
(6) the acupuncture point stimulation coil to constitute the zero magnetic field according to (5), wherein the support body is made of urethane or silicone.
(7) the acupuncture point stimulation coil to constitute the zero magnetic field according to any one of (5) to (6), wherein the support body is kneaded or coated with platinum silicone, carbon, or tourmaline.
(8) a method for using the acupuncture point stimulation coil to constitute the zero magnetic field, including a step of appropriately arranging at least one individual member to improve blood flow wherein the acupuncture point simulation coil includes a single conductive wire continuously extending, and the at least one individual member, each having a spiral portion wound in a left-winding direction and another spiral portion wound in a right-winding direction as one basic unit, the spiral portion and the another spiral portion being vertically combined.

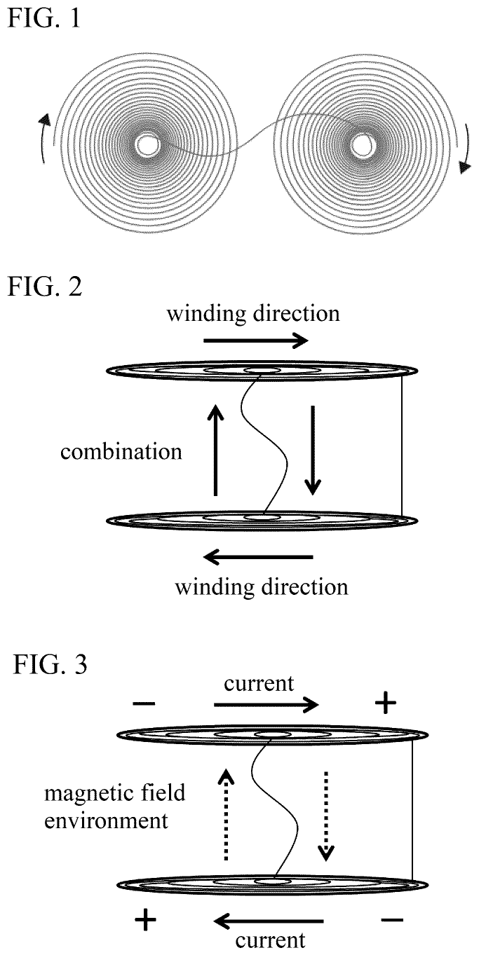

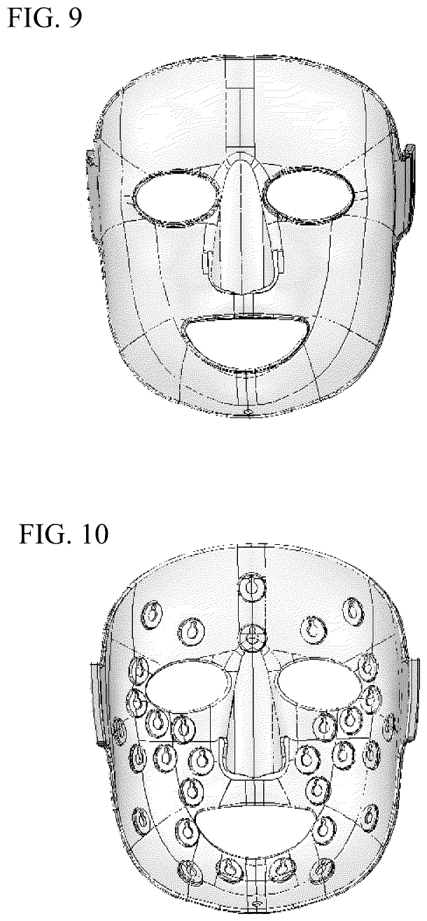

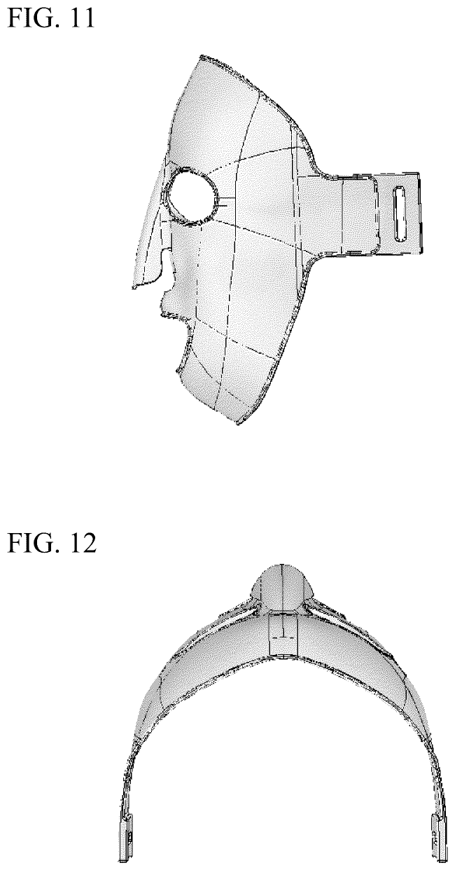

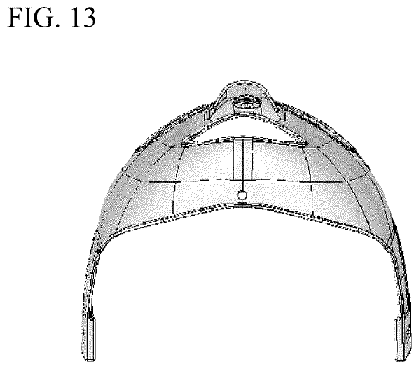

As disclosed above, the acupuncture point stimulation coil to constitute the zero magnetic field, includes a single conductive wire and individual members, each having a spiral portion wound in a left-winding direction and another spiral portion wound in a right-winding direction, the spiral portion and the another spiral portion being vertically combined to be one unit; and in an independent type, one individual member is applied to one acupuncture point, and two individual members are included in the coil or two or more individual members are continuously included in the coil. The basic structure and method are shown in FIG. 1. A size of the coil is generally 9 mm to 20 mm in diameter and appropriately selected depending on the intended use. If more than two individual members are arranged, it may be manufactured by repeating combination of the basic units. A distance between the individual members may be arbitrarily determined to be along the acupuncture point by adjusting a length of the conductive wire. The product designed as an independent type with a single coil is the face mask shown in FIGS. 9 to 13.

The upper and lower coils combined with each other are in spiral states wherein the winding directions are opposite to each other; therefore, currents are generated by biological current in opposite directions and magnetic fields are generated by the currents in opposite directions. Then, these currents and phenomena of the magnetic fields are interacted each other; as result, an equilibrium state is caused and a zero magnetic field is constituted. The areas around the coil is approximated as the zero magnetic field; however, the stimulation due to the pulse wave currents microscopically affects at the usage areas. Therefore, if the coil is arranged at the acupuncture point, blood flow is appropriately maintained, and if blood retention occurs, it is improved and the blood flow is normalized.

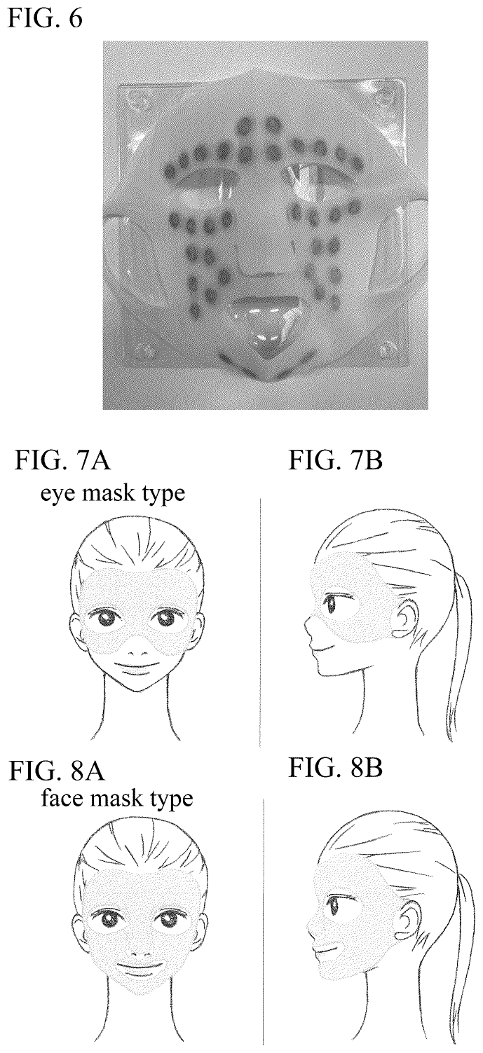

Products manufactured based on this mechanism and theory are the eye mask shown in FIG. 5 and the face mask shown in FIG. 6. The face mask is arbitrarily used in a separate type in which there are an upper mask upwardly extending from a nose and a lower mask extending from a lower portion of a lip to a neck. Further, from a standpoint of ease in processing, thickness and type of the support body are arbitrarily selected depending on arrangement of the coils and the number of the coils.

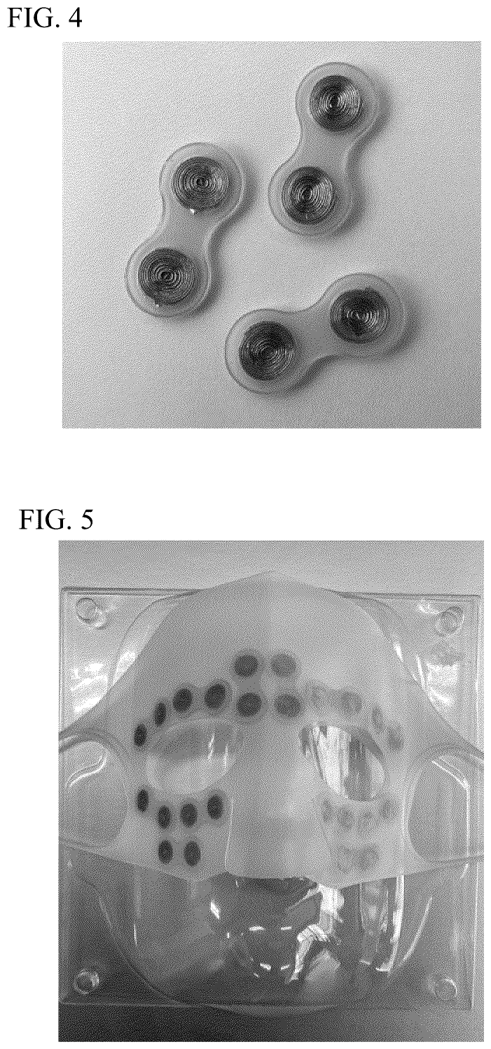

In the case that the coil in the present disclosure is partially used, the coil including one basic unit may be used. Additionally, the coil including two basic units as shown in FIG. 4 is appropriately used. In other words, the coils are arranged on the areas or the acupuncture points where people feel fatigue, to normalize the blood flow and to relieve the fatigue. It is appropriately applied to a nasal cavity to improve nasal congestion, hay fever, snoring etc. As other examples, the coil is partially used in a low back pain belt, a special supporter, and breast augmentation. Particularly, in the case that the breast augmentation is attempted to be achieved with improvement of blood flow, the coils are arranged around a brassiere. Similarly, the products including the coils are appropriately applied in order to improve the blood flow in an abdomen, wrists, etc. Furthermore, it is expected to increase metabolism (diet effect) with the improvement of the blood flow in an entire body by appropriately arranging the coils entirely, and for example, products such as a diet suit and a diet supporter for a specific part are provided.

As a countermeasure against acne, the coil is applied to acne bacteria in order to have antibacterial effects. Particularly, the conductive wire made of copper achieves dual effects of antibacterial and improving the blood flow. A puff type and a mask type are preferably used.

There are many cases in which lower limbs and toes suffer from health problems such as so-called “swelling” due to an abnormal balance between water content and blood flow. In this case, the product including the coil is used to improve the blood flow and the like. For example, there are embedded types in which the coil is embedded in an insole of athletic shoes, a counter at an internal tip, a pump lining, a quarter lining near a heel, etc. In a supporter type, the coil is embedded in a supporter for an elbow, a knee, a toe, and a heel, and socks and gloves types are arbitrarily produced as a countermeasure against cold feet.

As products for sleeping, there are a neck stall and a pillow for peaceful sleeping, and a bed mat for improving blood flow in an entire body. For example, a mat in which the a coil is sewn or attached is placed under a pillow or a pillow cover and the other mat in which the coil is sewn or attached is placed between a bed mat and a bed sheet, and two mats are arbitrarily combined to be used. Additionally, there are other products for a lift-up band, a hair band, a headband type, and a pendant type. There are a neckband and a stiffened shoulder pad for improving neck stiffness and shoulder stiffness, besides the supporter type. A nightcap is expected to eliminate static electricity. As disclosed above, the static electricity is a negative factor to prevent the blood flow in the human body, and the static electricity is easily generated in a hair due to friction while sleeping; therefore, it is very valuable to wear the nightcap to prevent the generation of the static electricity.

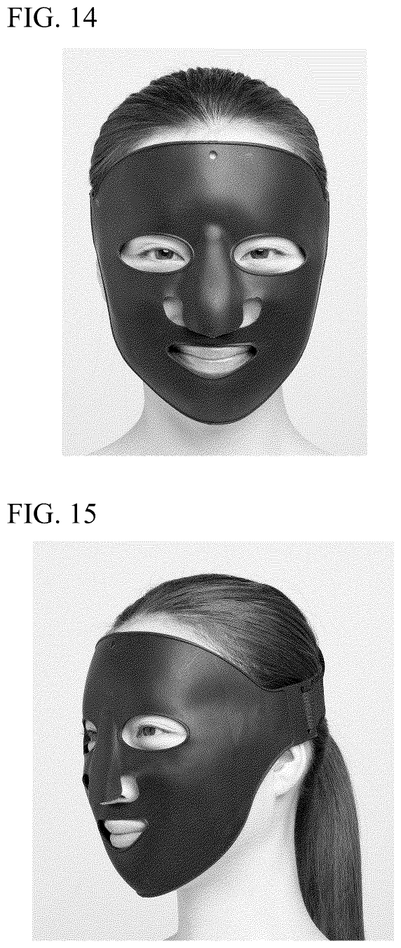

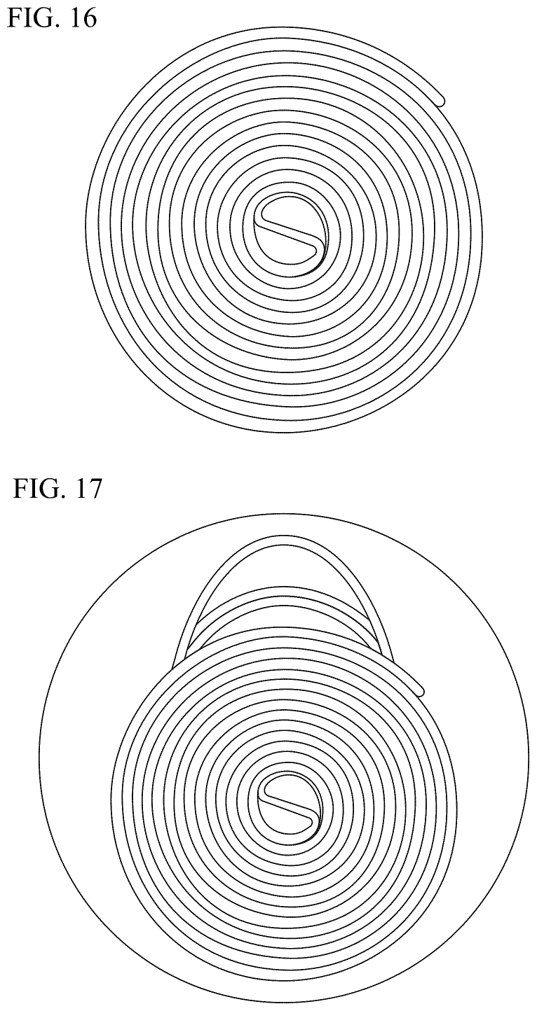

As disclosed above, the zero magnetic field coil in the present disclosure has effects of improving and normalizing the blood flow; therefore, it has excellent versatility to be applied in any daily life situations. As one example, trial products of the eye mask and face mask are shown in FIGS. 5 and 6; however, each of the eye mask and face mask is shown as a skeleton type to easily understand arrangement of the zero magnetic field coil. In fact, generally, it is produced into an opaque black type by blending functional materials having antibacterial properties such as carbon and tourmaline besides platinum silicone; however, it may be produced into white color type by adding titanium oxide to have a photocatalytic function for antibacterial purpose. FIGS. 7 and 8 show examples of fitting the eye mask on eyes and mounting the face mask on the face; however, the ear hook part may be produced to arbitrarily adjust the position or the number of holes because there are individual differences in the areas of the face. Further, it may cause an issue with elasticity or strength depending on the material of the sheet and the ear hook type may not be suitable; therefore, for example, a band fitting type is appropriately used (FIGS. 9 to 15). In production efficiency, it is most preferable to embed the coil into the support body, and FIG. 17 shows a schematic diagram of its example. The number of coils and forming types may be arbitrary selected unless the purpose of the present disclosure is not impaired, as disclosed in each above example.

### <Performance Test 1> Action for Increasing an Amount of Blood Flow

### (1) Test Outline

It was confirmed that the sheet product with the zero magnetic field coil that is a source of the properties in the present disclosure has an effect of increasing an amount of blood flow. Amounts of the blood flow before applying the test sheet to a face and during a certain period of time (1 minute, 3 minutes, and 5 minutes after removing the test sheet) after applying the test sheet for 15 minutes were measured 5 times each and the amounts of their changes were compared.

### (2) Measurement Result and Consideration

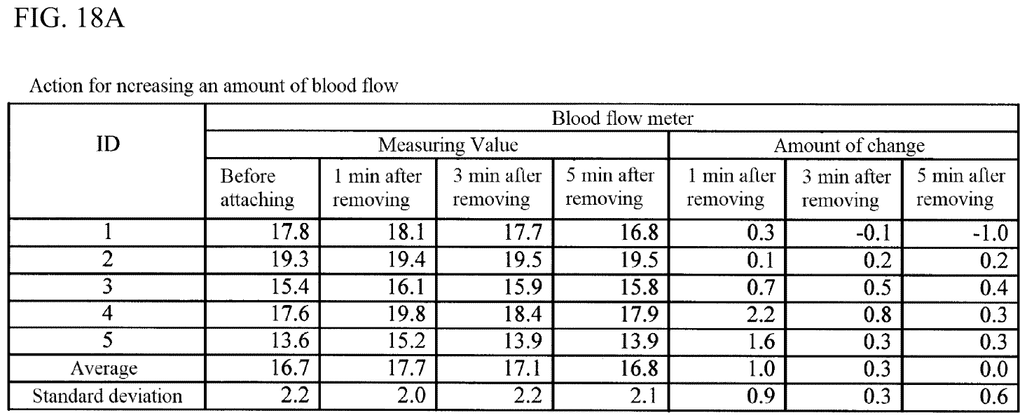

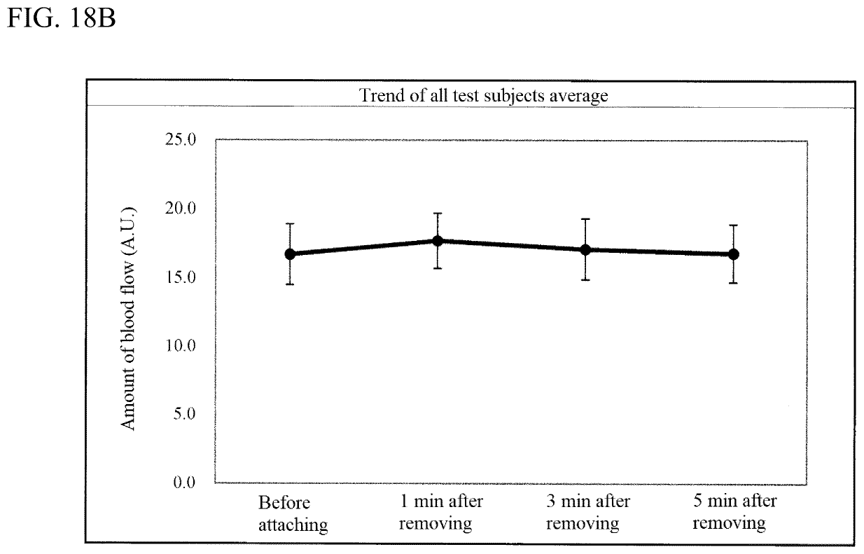

The result is shown in FIG. 18. After the coil sheet was removed, the amount of the blood flow decreased overt time; however, the amount of the blood flow was maintained when it was measured at 1 minute after removing the test sheet. By applying the coil sheet to a skin for a long time, it is expected to stably increase the amount of the blood flow.

### <Performance Test 2> Action for Suppressing Positive Ions

### (1) Test Outline

As for a sheet product with the zero magnetic field coil that is a source of properties in the present disclosure, effect of suppressing positive ions was confirmed by comparing positive ion concentration with negative ion concentration. Background measurement values (measurement values of positive ions and negative ions without the coil) are used for comparison.

### (2) Measuring Method and Measurement Result

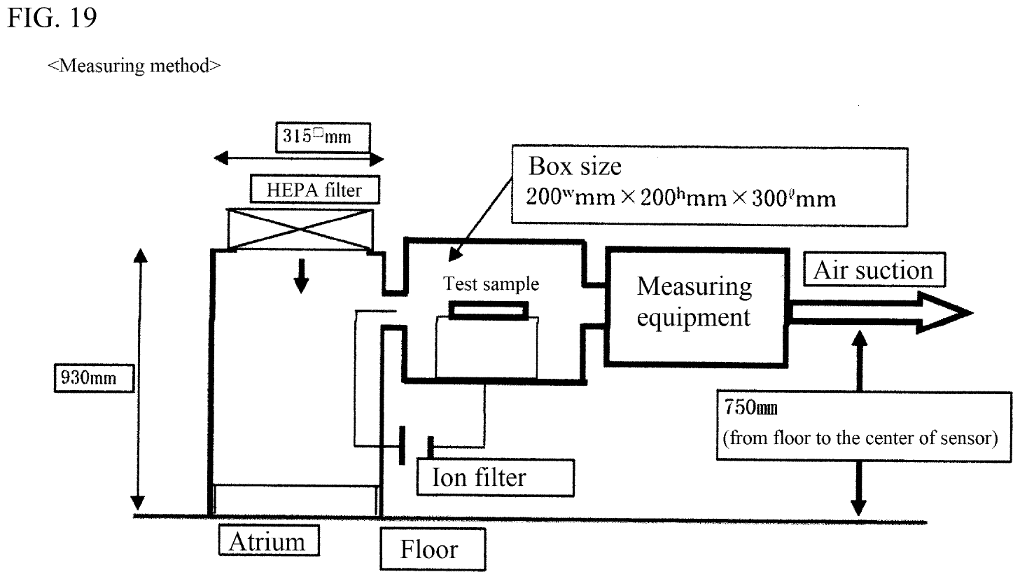

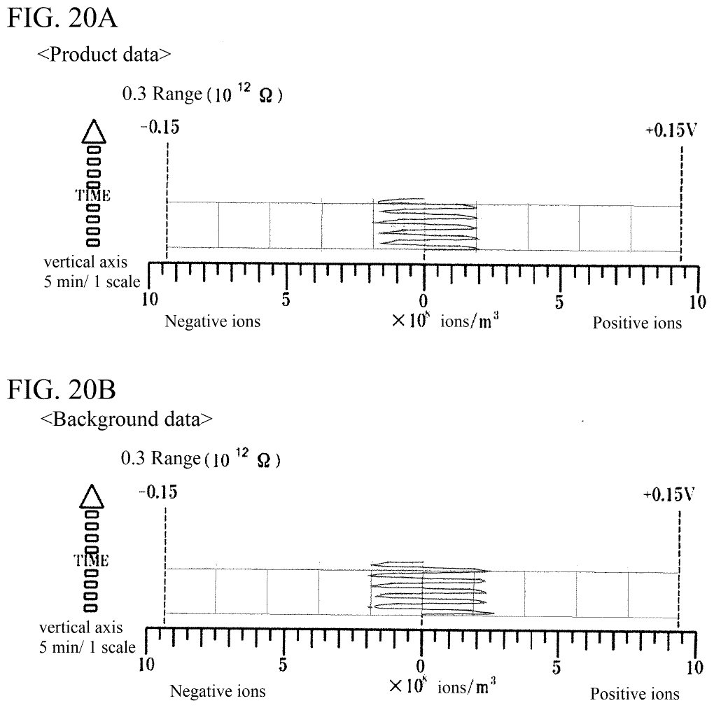

Test samples and their results are shown in Table 1. Measurement images and visual data of the results are shown in FIGS. 19 and 20.
Measuring equipment: Gerdien type ion counter
Measuring flow rate: 1.0×10⁻³ m³/s (60 L/min)
Threshold value of movement: 0.8×10⁻⁴ m²/sec·volt

TABLE 1

| Test shape | Sheet (Classified into powder, cloth, sheet, liquid, and others) | | | | |
|---|---|---|---|---|---|
| Measuring | Association standard equipment MODEL-SN | | | | |
| equipment | Main body structure Cylindrical capacitor | | | | |
| | Measuring parts Negative feedback type vibrating capacitance electrometer | | | | |
| Measuring | May 26, Reiwa 3(2021) | | | | |
| date | | | | | |
| Measuring | Measuring room in Japan Association of Ion Research and Application | | | | |
| place | | | | | |
| Weather | Cloudy | | | | |
| Room | 25° C. | | | | |
| temperature | | | | | |
| Humidity | 39% | | | | |
| Atmospheric | 1003 hpa | | | | |
| pressure | | | | | |
| Air | Class 5 classified by JIB B9920 | | | | |
| cleanliness | | | | | |
| Size | | | | | |
| Measuring | A box made by SUS was arranged at a tip end of a sensor, and a test | | | | |
| method | sample was placed inside the box and measured. Further, an air purifier | | | | |
| | (HEPA filter) and iron filter were arranged at a tip end of a tube on | | | | |
| | an air introduction side of the box. | | | | |
| Measurement | | Background | | Measuring value of product | |
| result | Positive ions | Average | 2.5 × 10⁸ ions/m³ | Average | 2.0 × 10⁸ ions/m³ |
| | | Maximum | 2.7 × 10⁸ ions/m³ | Maximum | 2.1 × 10⁸ ions/m³ |
| | | Minimum | 2.3 × 10⁸ ions/m³ | Minimum | 2.0 × 10⁸ ions/m³ |
| | Negative ions | Average | 2.0 × 10⁸ ions/m³ | Average | 1.9 × 10⁸ ions/m³ |
| | | Maximum | 2.1 × 10⁸ ions/m³ | Maximum | 2.1 × 10⁸ ions/m³ |
| | | Minimum | 1.8 × 10⁸ ions/m³ | Minimum | 1.7 × 10⁸ ions/m³ |
| | Evaluation | Positive ions | Ion density | | (Measuring value |
| | category | Negative | class | | of product- |
| | (evaluating only | ions | | | Background |
| | product) | | | | value) or |
| | | | | | evaluated by 3σ |
| | | | | | value of B.G |

### (3) Consideration

As clearly shown in the above results, no effect of increasing the negative ions (good environmental factor) was confirmed; however, the effect of lowering the background value was confirmed in the positive ions (bad environmental factor). Assuming that an index of the negative ion concentration is set as 1, the background index was reduced from 1.25 to 1.05 by using the product. As a result, it was implied that the balance of the positive ions and the negative ions in the living environment was improved.

### <Performance Test 3> Antibacterial Test

### (1) Test Outline

Firstly, the zero magnetic field coil in the present disclosure was sterilized before conducting the test. The method for sterilizing the coil included soaking the coil in 75% ethanol overnight, and irradiating both sides of the coil with ultraviolet rays for one hour to remove unwanted bacteria not killed by alcohol sterilization. In the test, 6 pieces of the zero magnetic field coils were attached to a test subject's left check for one hour, and it was repeated for three days. On the fourth day, 15 pieces of the coils were attached and left for 30 minutes (the adhesive surface between a skin and a tape was minimized by arranging the coils without any gaps on the tape to prevent the coils from peeling off due to the adhesive force of the tape). Thereafter, the coils were removed and the bacteria on the skin were collected by “contact plate method” and “wipe method.” In the “contact plate method,” medium plates for coliform, Staphylococcus aureus, and fungi were contacted with the test subject's both cheeks. The bacteria was cultured for 24 to 48 hours and colonies grown on the plate were counted. On the other hand, in the “wipe method,” after wiping the test subject's both cheeks with absorbent cotton soaked in 0.5 ml of extracted liquid, it was soaked in 5 ml of the extracted liquid, and the bacteria was extracted; thereafter, it was applied to each of the medium plates for general bacteria coliform, Staphylococcus aureus, and acnes bacteria, and the bacteria was cultured for 24 to 48 hours and the colonies grown on the plate were counted.

### (2) Test Result

In the “contact plate method,” no coliform, Staphylococcus aureus, and fungi was detected from the test subject's cheek to which the coil was attached; however, 6 colonies of Staphylococcus aureus and 2 colonies of fungi were detected per 10 cm² from the test subject's cheek. In the “wipe method,” the number of general bacteria and acnes bacteria were suppressed to approximately one-tenth on the test subject's cheek to which the coil was attached, and the number of Staphylococcus aureus was suppressed to less than one-hundredth. Additionally, as for the acnes bacteria, suppressing test was conducted on an agar medium. A culture solution of the acnes bacteria was applied to the anger medium, and 9 pieces of the coils were placed on the culture solution and left for one hour; thereafter, the coils were removed and cultured at 30° C. under anaerobic conditions, and colonies were detected one week later. As a result, in the plate medium wherein the coil was left for 1 hour, the grown colonies were small and it was implied that there was the possibility of suppressing the growth of the acnes bacteria.

### (3) Consideration

Staphylococcus aureus is rarely seen on healthy skin, and a large number of these bacterium are present on atopic skin. Further, it was recognized that Staphylococcus aureus causes dermatitis and acne besides atopic dermatitis. Therefore, it was implied that the coil in the present disclosure was effective to normalize the skin.

TABLE 2

| | Contact plate method | | Wipe method | |
|---|---|---|---|---|
| | CFU/10 cm² | | CFU/5 ml | |
| Bacterial | Test | After attaching | Test | After attaching |
| species | subject | the coil | subject | the coil |
| General bacteria | — | — | 290 | 31 |
| Coliform | 0 | 0 | 0 | 0 |
| Staphylococcus | 6 | 0 | 300 | 0 |
| aureus | | | | |
| Fungi | 2 | 0 | — | — |
| Acnes | — | — | 620 | 61 |

### <Performance Test 4> Skin Quality Improvement Test

### (1) Test Outline

The skin condition (cheek area) was investigated and analyzed under conditions wherein the test product of “a beauty mask equipped with the zero magnetic field coil in the present disclosure” was continuously used for 4 weeks by adult women who concern dryness and wrinkles and take care of it with daily simple care. The skin condition of the same test subject before using the test product was compared with the skin condition of the same test subject after using the test product for 4 weeks by visual inspection and image analysis with VISIA photography, and the results were evaluated (n=12).

“Image Analysis with VISIA Photography”
Equipment: VISIA Evolution (Canfield Scientific Inc.)
Photographing conditions: after washing the test subject's face, acclimatizing for at least 20 minutes in an environmental test room and taking photographs
Measuring parts: Half face on either left or right
Number of photographing: 1 time
Analysis items: Stains, wrinkles, pores, uneven color, porphyrin, hidden spots, melanin index, hemoglobin index
Statistical analysis: Effectiveness evaluation is comparison-in-group comparing values of 0 w with values of 4 w in each evaluation item. For statistical testing, p values were calculated with corresponding t-test.

### (2) Measuring Method and Result

In the image analysis (score) with VISIA photography at 4w, significant decreases in wrinkle score and uneven color score were found in the comparison with 0 w. No other significant change was found. Only items that statistically significant differences were found are shown in Table 3.

### (3) Consideration

The significant changes were found in winkles and uneven color in the cheek area; therefore, it was implied that the coil in the present disclosure promoted the improvement of the blood flow and the normalized self-regeneration function.

TABLE 3
Results of Image Analysis by with VISIA Photography (score)

| | Winkle score | | | Uneven color score | | |
|---|---|---|---|---|---|---|
| | | | Amount | | | Amount |
| | | | of | | | of |
| | Measuring value | | change | Measuring value | | change |
| | (A.U.) | | (A.U.) | (A.U.) | | (A.U.) |
| | 0 w | 4 w | 4 w | 0 w | 4 w | 4 w |
| Average | 0.458 | 0.360 | −0.098 | 0.143 | 0.122 | −0.021 |
| Standard | 0.242 | 0.272 | 0.133 | 0.054 | 0.064 | 0.030 |
| deviation | | | | | | |
| comparison- | — | 0.026 | — | — | 0.276 | — |
| in-group | | | | | | |
| p value (*) | | | | | | |

A.U.: Arbitrary unit for eigenvalue of measuring equipment
(*) Corresponding t-test

### <Performance Test 5> Moisture Content in Keratin Test

### (1) Test Outline

The skin condition (cheek area) was evaluated by measuring moisture content in keratin under conditions wherein the test product of “a beauty mask equipped with the zero magnetic field coil in the present disclosure” was continuously used for 4 weeks by adult women who concern skin condition, especially moisture retention, and take care of it with daily simple care.

### “Measuring Moisture Content in Keratin”

Equipment: Corneometer (Courage+Khazaka electronic GmbH)
Measuring Conditions: after washing the test subject's face, acclimatizing for at least 20 minutes in an environmental test room and evaluating
Measuring Parts: Half face on either left or right
Evaluation Method: the evaluating parts were measured five times, and the average value in three measurements without the highest and lowest values was adapted.
However, if an abnormal value was measured, by decision of the person in charge of the test, the value was removed and re-measured.
Statistical Analysis: Effectiveness evaluation is comparison-in-group comparing values of 0 w with values of 4 w in each evaluation item. For statistical testing, p values were calculated with corresponding t-test.

### (2) Test Result

In the moisture content in keratin at 4w, significant increase was found in comparison with 0 w (p<0.001). The result was shown in Table 4.

### (3) Consideration

In the maintenance of the homeostasis in humans, the moisture retention function is important, and as considering the positive effect to the blood flow due to the moisture retention, it was confirmed that the zero magnetic field coil in the present disclosure is effective to solve the problems through the tests. If the balance between the moisture content and the blood flow is lost, it is clearly reflected in daily health conditions such as swelling; therefore, it was implied from the test results that the zero magnetic field coil in the present disclosure is effective to improve the swelling.

TABLE 4
Moisture content in keratin

| | Measuring value | | Amount of change |
|---|---|---|---|
| | (A.U.) | | (A.U.) |
| | 0 w | 4 w | 4 w |
| Average | 53.4 | 60.5 | 7.1 |
| Standard deviation | 12.8 | 10.5 | 4.1 |
| comparison-in-group | | <0.001 | |
| p value (*) | | | |

A.U.: Arbitrary unit for eigenvalue of measuring equipment
* Corresponding t-test

1. An acupuncture point stimulation coil to constitute a zero magnetic field, comprising:
individual members, each having a spiral portion wound in a left-winding direction and another spiral portion wound in a right-winding direction, the spiral portion and the another spiral portion being vertically combined; and
at least one conductive wire continuously extending, each connecting two of the individual members,
wherein at least two individual members are continuously arranged through the at least one conductive wire.

2. The acupuncture point stimulation coil according to claim 1, wherein the at least one conductive wire is made of copper or aluminum.

3. The acupuncture point stimulation coil according to claim 1, further comprising a silicone cover covering the individual members connected through the at least one conductive wire.

4. The acupuncture point stimulation coil according to claim 3, wherein the silicon cover is kneaded or coated with platinum silicone, carbon, or tourmaline.

5-7. (canceled)

8. A method for using the acupuncture point stimulation coil to constitute a zero magnetic field, comprising:
a step of appropriately arranging at least one individual member to improve blood flow,
wherein the acupuncture point simulation coil includes
the at least one individual member,
each having a spiral portion wound in a left-winding direction and another spiral portion wound in a right-winding direction, the spiral portion and the another spiral portion being vertically combined to be one unit, or
each having at least two spiral portions wound in the left-winding direction and at least two another spiral portions wound in the right-winding direction, the at least two spiral portions and the at least two another spiral portions being vertically combined, respectively, to be at least two units, and
at least one conductive wire continuously extending, each connecting at least two units.

9. An acupuncture point stimulation coil to constitute a zero magnetic field, comprising:
an individual member configured to be arranged at an acupuncture point and having a spiral portion wound in a left-winding direction and another spiral portion wound in a right-winding direction, the spiral portion and the another spiral portion being vertically combined.

10. The acupuncture point stimulation coil according to claim 9, further comprising a silicone cover covering the individual member.

11. The acupuncture point stimulation coil according to claim 10, wherein the silicon cover is kneaded or coated with platinum silicone, carbon, or tourmaline.

12. An acupuncture point stimulation coil structure, comprising:
a support body having a shape along acupuncture points,
an acupuncture point stimulation coil to constitute a zero magnetic field, including
first individual members, each having
a spiral portion wound in a left-winding direction and another spiral portion wound in a right-winding direction, the spiral portion and the another spiral portion being vertically combined to be one unit, or
second individual members, each having
at least two spiral portions wound in the left-winding direction and at least two another spiral portions wound in the right-winding direction, the at least two spiral portions and the at least two another spiral portions being vertically combined, respectively, to be at least two units, and
at least one conductive wire continuously extending, each connecting two of the two units,
wherein a predetermined number of the first individual members is randomly arranged on the support body, or the second individual members are arranged corresponding to the shape of the support body.

13. The acupuncture point stimulation coil structure according to claim 12, wherein the support body is made of urethane or silicone.

14. The acupuncture point stimulation coil structure according to claim 12, wherein the support body is kneaded or coated with platinum silicone, carbon, or tourmaline.
